(19) United States
(12) Patent Application Publication
(10) Pub. No.: US 2022/0104996 A1
(43) Pub. Date: Apr. 7, 2022

(54) WIRELESS INDUCTIVE POWER AND CONTROL OF BODY SURFACE STIMULATORS

(71) Applicants: Jeff BENNETT, Maple Grove, MN (US); Michael HOEY, Maple Grove, MN (US); Roger HASTINGS, Maple Grove, MN (US); Shawn MCCUTCHEON, White Bear Lake, MN (US)

(72) Inventors: Jeff BENNETT, Maple Grove, MN (US); Michael HOEY, Maple Grove, MN (US); Roger HASTINGS, Maple Grove, MN (US); Shawn MCCUTCHEON, White Bear Lake, MN (US)

(21) Appl. No.: 17/495,740

(22) Filed: Oct. 6, 2021

Related U.S. Application Data
(60) Provisional application No. 63/088,373, filed on Oct. 6, 2020.

(51) Int. Cl. A61H 19/00; H02J 50/10

(52) U.S. Cl. CPC A61H 19/50; H02J 50/10; A61N 1/3603; A61H 2201/5097; A61H 2201/165

(57) ABSTRACT

A system having one or more remote sexual wellness devices and control devices. The control devices are configured to activate an action mechanism of the remote sexual wellness device. Activation of the action mechanism can be controlled by one or more parameters. The system also includes a remote powering or charging system to power or charge the sexual wellness devices and control devices.

### PRIORITY

This application claims the benefit to U.S. Patent Application No. 63/088,373 filed on Oct. 6, 2020, the entirety of which is incorporated herein by reference.

### FIELD

The present invention relates generally to the wireless charging, powering, activation, and control of generally remote devices.

### BACKGROUND

A number of devices and methods are available for controlling remote devices. These devices typically utilize conventional means of communication such as infrared, radio control, and more recently, wireless fidelity and cellular. All of these devices and methods, however, fail to provide a means of providing remote powering.

Conventional devices and methods have to be charged by a cable or a device that uses a low range inductive charging system such as Qi wireless charging. Devices requiring a cable to charge a device often result in the device being unusable or at least greatly restricts a user's ability to use the device while it is being charged. As a result, the lengths of charging cables have been growing considerably.

Qi wireless charging works by placing a device needing to be charged upon a surface of another device capable of charging by induction. This technology was intended to revolutionize the process of charging by eliminating the cable. However, since a device needing to be charged must lay generally flush with a surface of the charger device, it has resulted in an even greater restriction on the use of the device during charging.

As a result, there is still a need for devices and methods capable of charging or powering a remote device. There is also a need for devices and methods capable of charging or powering a remote device while is also being used or remotely controlled.

### BRIEF DESCRIPTION OF THE DRAWINGS

FIG. 1 is a diagram view of a remote power and control system for sexual wellness devices according to an example embodiment.

FIG. 2A is side view of a sexual wellness device bra according to an example embodiment of the invention.

FIG. 2B is front view of a sexual wellness device panty according to an example embodiment of the invention.

FIG. 3 is a diagram view of a remote power and control system for sexual wellness devices including outer garments and under garments that are activated and controlled by proximity to each other.

FIG. 4 is a perspective view of sexual wellness devices consisting of intimate personal devices that are activated and controlled by proximity to each other.

FIG. 5 is a schematic diagram of a circuit of the system according to an example embodiment of the invention.

FIGS. 6A and 6B are illustrations of antennae according to an example embodiment of the invention.

FIG. 7 is an illustration of coil antennae according to an example embodiment of the invention.

FIG. 8 is a chart depicting power coupling efficiency vs separation between transmit and receiver coils according to an example embodiment of the invention.

FIG. 9 is an illustration of a sexual wellness device placed on a user according to an example embodiment of the invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the scope of the invention as defined by the appended claims.

### DETAILED DESCRIPTION OF THE INVENTION

The present invention includes system 10 for powering and controlling sexual wellness devices 12. One such sexual wellness device 12 includes a battery powered nerve stimulation patch invented by Morari Medical, Inc. (“Morari”). The stimulation patch is used for improving sexual wellness by delaying an ejaculatory event during intercourse. The device is also capable increasing a low libido in women. The Morari patches may be controlled from a remote control or an application on a cellular phone 14. The communication between the remote control or cell phone 14, the sexual wellness device 12, and the rest of the system 10 can be accomplished via Bluetooth® or other communication protocol.

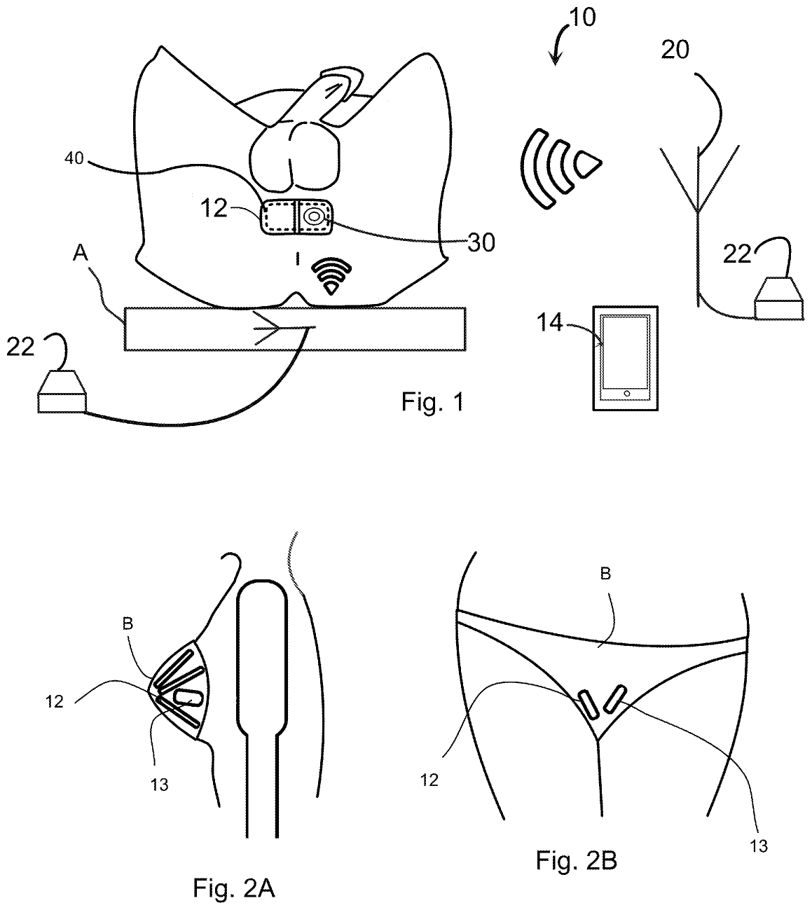

As illustrated in FIG. 1, one aspect of the present invention is a wireless charging and control system 10 that includes remote or long range-inductive powering antennae 20, placed on, proximate to, adjacent to, or in a location generally near an individual's body, to charge a remote sexual wellness device 12 such as the Morari stimulation patch. The antennae 20 can be placed on a piece of furniture or coupled to a room surface such as a wall or a floor. In one example embodiment, antennae 20 is contained within or under a mattress pad or other portion of a piece of furniture such as a chair or sofa cushion. It is also contemplated herein that antennae 20 can be located under the bed or in any location where powering or controlling a remote sexual wellness device 12 is desirable.

Power can be delivered antennae 20 by any means. For example, antenna 20 can be plugged directly into a wall outlet or battery. In one embodiment of the invention, a control box or controller 22 is provided that is in operative communication or connected to antenna 20. Control box or controller 22 can be located generally near to or remote from antennae 20. For instance, as illustrated in FIG. 1, when antennae 20 is placed in or under a mattress pad A, control box or controller 22 can be placed near or under the bed. The control box or controller 22 can then be connected to a power source such as a wall outlet or battery.

A variety of remote sexual wellness, stimulation and therapy devices 12, including wireless stimulation patches, are disclosed herein. As illustrated in FIG. 1, the sexual wellness device 12 can use electrical stimulation to stimulate nerves in the pelvic or groin area of a user to treat pelvic disorders such as premature ejaculation. It can also be used to stimulate the nerves of the penis in order to assist men in achieving an erection. As illustrated in FIGS. 2B and 4, the sexual wellness devices 12 can also be used to electrically stimulate the nerves of women in order to improve their sexual wellness and/or satisfaction.

While electrical stimulation is one aspect of the sexual wellness devices 12 of the present invention, other types of stimulation and therapy as also contemplated herein. As illustrated in FIGS. 2A-2B and 2B, sexual wellness devices 12 can have or include a stimulation mechanisms or contraction mechanisms 13 (utilizing electropolymers and the like) that stimulate a user by physical contractions or some other non-electrical means.

Continuing with FIGS. 2A and 2B, sexual wellness device 12 can be incorporated into any number of clothing articles. For instance, sexual wellness device 12 (either stimulation or contraction mechanisms) can be incorporated into any undergarments such as bras or underwear B. Any undergarment may be used with the present invention and the presented examples should not be considered limiting.

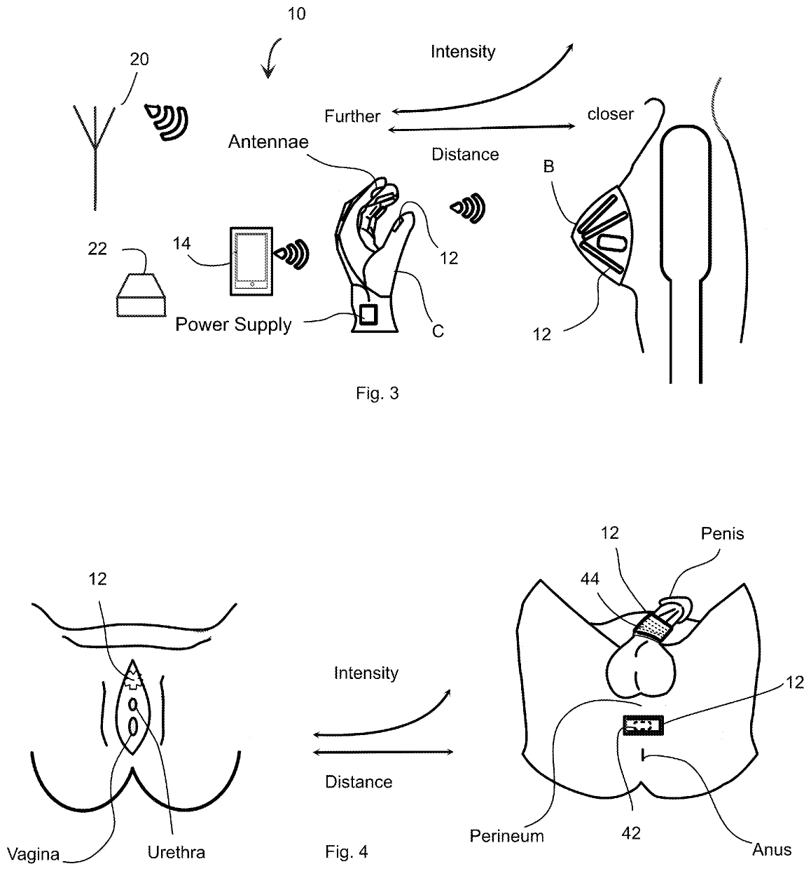

Sexual wellness device 12 can also be incorporated into outwear such as gloves, shirts, pants, socks, shoes, bracelets, anklets, headwear and any other types of clothing and accessories. As illustrated in FIG. 3, sexual wellness device 12 (with our without contraction mechanism 13) is incorporated into a glove C. Any portion of glove C is able to support sexual wellness device 12. FIG. 13, for example, illustrates one or more types of sexual wellness device 12 positioned on the figure tips of glove C. Positioning one or more sexual wellness devices 12 in this manner enables a user to use glove C by themselves or with a partner. Further, glove C or similar garment enables a user to stimulate various body parts or regions by simply moving their hand.

Continuing with FIG. 3, outer garments and inner garments can be used together to provide an elevated multi-person experience. For example, an outer garment, such as glove C, having or being controlled by a remote control 22 and/or transmitter/receiver 14, can be used with one or more inner garments such as a bra and/or panty B, having one or more sexual wellness devices 12 incorporated therein. In this arrangement, glove C is able to transmit and control one or more sexual wellness devices 12 incorporated in an undergarment.

Referring to FIG. 4, two or more sexual wellness devices 12 can be used, with each sexual wellness device 12 having a controller 22 and/or transmitter/receiver 14 that is able control or respond to the other sexual wellness devices 12. For example, one or more sexual wellness devices 12 can be placed on one partner and one or more sexual wellness devices 12 can be placed on another partner. Sexual wellness devices 12 on each partner are then able to control, interact with, or respond to each other by causing various actions/reactions that can be random or predetermined.

The various actions can include, but are not limited to, electrical stimulation, vibration, mechanical massaging, tightening or constricting, loosening, heating, cooling, or any combinations thereof. Other actions are possible and may be programmed by the users or downloaded from an internet site.

The various actions between controllers 22, transmitters/receivers 14, and sexual wellness devices 12 or between multiple sexual wellness devices 12 can be controlled by various means. For instance, an action can be triggered or actuated by adjusting or changing a distance between the controllers 22, transmitters/receivers 14, and sexual wellness devices 12. In another instance, the distance between a controllers 22, transmitters/receivers 14, and sexual wellness devices 12 or between the sexual wellness devices 12 can increase or decrease an intensity of an action of sexual wellness devices 12. For example, if the distance between the controllers 22, transmitters/receivers 14, and sexual wellness devices 12 or between the sexual wellness devices 12 is increased, the intensity can decrease or decrease. It is also contemplated herein that movement of a controller 22, such the undergarments or outer garments, can control different actions of sexual wellness devices 12.

FIGS. 3, 4 and 9 provide illustrated examples of the above single and multi-partner use of sexual wellness devices 12. As illustrated, one or more sexual wellness devices 12 (e.g., personal massagers and/or stimulators) can be placed on one or both partners. The placement of sexual wellness devices 12 varies and can be chosen by the user or users. Example placements include on a i) shaft of a penis, ii) above, below or on the sides of a penis, iii) on, about or adjacent to the scrotum, iv) on any part or portion of a vagina and/or clitoris, v) around or in the vaginal canal, and vi) on, in or around a user's anus or rectum. Any location desired by a user is contemplated herein and should not be considered limiting. It is also contemplated herein that users are able to program the garments and/or sexual wellness devices 12 to perform any desired action as a result of any particular event.

It is contemplated herein that therapists can prescribe the present invention as a method of counseling partners. For example, a therapists can advise a couple to use sexual wellness devices 12 and/or the garments having or incorporating sexual wellness devices 12 as an aid to improve intimacy and/or love and affection. Since sexual wellness devices 12 are able to be programmed or preprogrammed, a user can set it to whatever is arousing or preferred by them. Sexual wellness devices 12 can also be programmed to cause an action (contraction and/or stimulation), such as genital stimulation, to occur at a particular time or during an event that is generally difficult for a couple. For instance, if a couple is having a difficult time being near each other, sexual wellness devices 12 can be programmed to stimulate the respective couple as they draw nearer to each other. In this way, the couple is stimulated or rewarded for being near or approaching each other. It is believed that this will increase arousal and attraction between the couple.

While the action of a sexual wellness device 12 has been described in detail as stimulation it can also include vibration, movement, contraction, expanding, increase/decrease in pressure, and the like. Similarly, although an event causing an action has been described as proximity of the sexual wellness devices 12, it can include any event programmed into the device. For instance, a particular time or day can be a triggering event. Sexual wellness devices 12 of the present invention can also include sensors that can be used to monitor for a triggering event. For instance, sexual wellness devices 12 can include microphones or light sensors that are able monitor for a particular sound, word, or any light or darkness. Upon detecting a chosen word, sound, light, or darkness, one or both sexual wellness devices 12 can be activated. Any sensor and any triggering event may be used and can be programmed into controller 24, transmitter/receiver 14, and/or sexual wellness devices 12.

Referring back to FIG. 3, sexual wellness devices 12 can be a generally self-contained unit having one or more power supplies and one or more antennae 20. One sexual wellness device 12 can also be passive with power being supplied by other sexual wellness devices 12. It is also contemplated herein that an external power and/or control box 22 and antennae 20 can be used to provide power to one or both of the sexual wellness devices 12.

Sexual wellness devices 12 can also include one or more power storages or batteries to store power for later usage. Either an external antennae 20 or an internal antennae 20 of a sexual wellness device 12 can supply power, via resonant inductive coupling, to one or more other sexual wellness devices 12 that are within a charging distance.

Resonance is achieved by tuning each antenna 20 with a capacitor to a specific frequency. As illustrated in FIG. 1, power is transmitted by an antennae 20 to one or more small coils 30 in sexual wellness devices 12, controllers 22, and/or transmitter/receiver 14 that are tuned to the same frequency.

Power coupling efficiency can be high enough to power stimulation circuits provided that the tuning quality factor, Q of both the transmit and the receive circuits is relatively large. The average power needed for surface electrical stimulation, for example, is in the range of milli-Watts, while antennae 20 can generate Watts of power. Effective stimulation can thereby occur even when the power coupling efficiency is low, and even when there are multiple stimulators receiving power. The apparatus or system 10 of the present invention is designed so that the highest stimulation power generates local AC magnetic fields that are safe for human exposure.

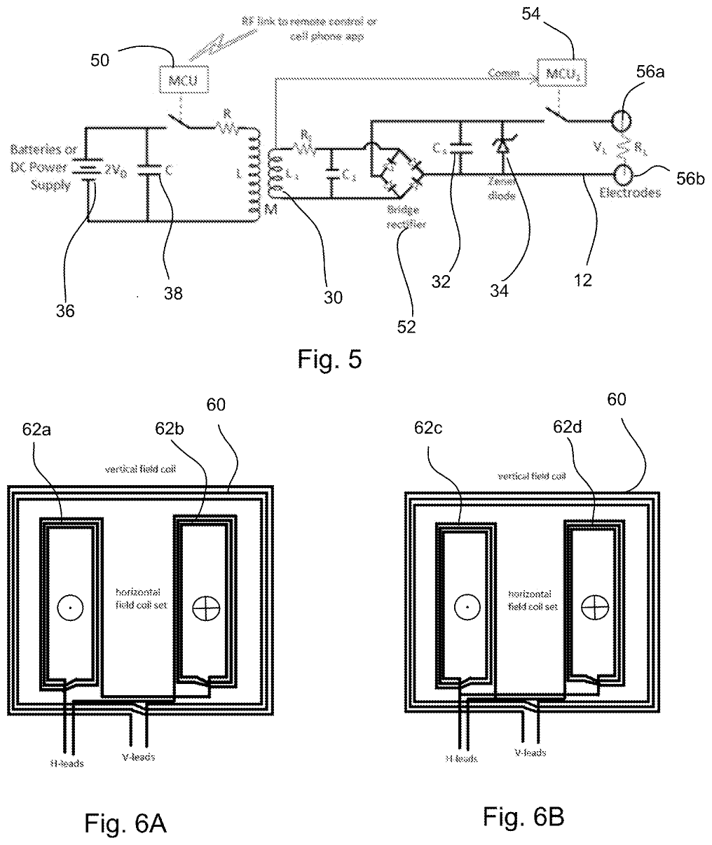

Referring to FIG. 5, power for sexual wellness devices 12, such as the Morari stimulation patch, is received in a small wire coil 30, the output of which is rectified and stored on a capacitor 32. Stimulation maximum voltage can be set by a passive voltage control element such as a Zener diode 34. Alternatively, a microcontroller can control voltage and other stimulation parameters. Electronics components, including the microcontroller, are miniaturized to fit comfortably in the area of sexual wellness devices 12. Stimulation or other action commands may be passed to the on-board microprocessor via the inductive link.

As discussed above, the actions of sexual wellness devices 12, such as garments worn by a user or sexual wellness devices 12 placed directly on a user's body, are stimulated to activate touch receptors in the skin of the user to promote arousal. Film electrodes 40 (broken line of FIG. 1) can be integrated into any of sexual wellness devices 12 to provide electrical stimulation, vibrations and the like. The apparatus or system 10 of the present invention can also include a conductive gel 42 (broken line of FIG. 4) that may be applied to the skin to promote electrical contact with electrodes of the sexual wellness devices 12. As mentioned above, sexual wellness devices 12 may contain electro-active or magneto-active fibers or polymers 44 (broken lines of FIG. 4) that contract, expand or otherwise move/change when energized or de-energized by the received inductive power.

Portions or parts of sexual wellness devices 12 may be stimulated sequentially to provide waves or patterns of stimulation that promote sexual arousal. It is also contemplated herein that garments can have multiple sexual wellness devices 12 with each sexual wellness device 12 being able to be individually or sequentially controlled to cause or increase arousal. Controlling multiple sexual wellness devices 12 in a garment can provide an increased area of stimulation or arousal. It is also contemplated herein that the garment is able to simulate a user by human touch (with or without a controller 22, such as another garment). Touch sensors can be incorporated into sexual wellness devices 12 to react to pressure or movement associated with touching or some physical interaction.

To accomplish the activation and control of a garment having sexual wellness devices 12, transmit antennae 20 may be integrated into one partner's garment sexual wellness device 12 or sexual wellness devices 12, while a receive circuit is integrated into the other partner's garment having a sexual wellness device 12 or sexual wellness devices 12. As described above, examples may include transmit coils (antennae) in a glove C and receive circuits proximate selected erotic zones of the partner. This application may take advantage of the increase in stimulation intensity as transmit and receive circuits approach one another, thereby mitigating the need for voltage control circuits in the receive circuit.

While the invention has been described as a sexual wellness device, it is also contemplated that devices 12 also have therapeutic properties that are beneficial to patients. For instance, devices 12 can include stimulation applications, including TENS electrodes that may be wirelessly stimulated with no electrical leads. The wireless devices 12 increase comfort and mobility for patient/user while sleeping, sitting in a chair that contains stimulation antennae 20, for example in a pad or blanket, or while moving about. When antennae 20 and devices 12 are integrated into garments or blankets that are worn by the patients/users they can be energized by a battery control 22 unit in a pocket of a garment or attached to a belt, for example. The patient/user can be mobile and active while being stimulated, without the discomfort and logistics of multiple leads connecting the devices 12 to the controller 22.

It is also contemplated herein, that the system and apparatus 10 of the present invention can include the ability to have charging stations in homes, nursing homes, and hospitals where a patient/user can go to have one or more remote patient/sexual wellness devices 12 charged, activated, and/or controlled. The present invention eliminates the need to have vulnerable patients carry power sources.

### Wireless Stimulation Circuitry

An example inductive power stimulation circuit is shown in FIG. 5. The primary R, L, C circuit is energized with pulses of DC current created at frequency f by microcontroller MCU switch closures. The power supply 36 may comprise batteries, which may be primary or rechargeable, or a wall powered DC power supply. The resistance R is primarily due to the resistance of transmit antenna coil L, and also due to resistance in capacitor C and any leads or board traces. If the frequency f is higher than about 100 kHz, the transmit antenna may comprise a Litz wire or equivalent to minimize circuit resistance.

As particularly illustrated in FIG. 5, operation of the circuit of the present invention includes pulses of current from a DC power source voltage 2V₀ are created by microcontroller MCU 50. Microcontroller MCU 50 commands switch closures that energize the primary R, L, C circuit at tuned pulse frequency f, which converts to sine waves of amplitude VO in inductance L. The primary L and secondary L1 are coupled through mutual inductance M to the secondary R1, L1, C1 circuit, also tuned to frequency f. Capacitor C1 38 discharges through a bridge rectifier 52 onto storage capacitor Cs 32. The DC charge stored on Cs 32 is dumped through tissue resistance RL through skin patch electrodes when the secondary switch is closed by MCU1 54 at a preferred tissues stimulation rate. MCU 50 communicates with MCU1 54, for example via interrupts in transmit power, to control pulse rate and other parameters.

### Transmit Antennae

An example of a transmit antennae coil 60 set is shown in FIG. 6A. The large multi-turn coil covers a desired area in the transmit pad, and projects a magnetic field vertically up out of the page. The pair of inner coils 62a and 62b are connected so that at the center of the left-hand coil 62a the magnetic field is vertically upward, while at the center of the right-hand coil 62b the magnetic field is vertically downward. In the space between the two coils the field is horizontal, directed from the right to left or left to right depending upon the current direction. A similar pair of coils placed at the top and bottom of the pad will produce magnetic fields that are in the plane of the figure directed upward or downward in the region between these coils. The combination of two horizontal and one vertical coil allows magnetic fields to be generated in any direction in space to optimize coupling to receive coils that may be directed at unknown angles relative to the transmitter.

The parallel electrical connection of the two inner horizontal coils 62c and 62d of FIG. 6B may be preferred over the series connection of FIG. 6A. This is because the parallel connection has twice the current and twice the magnetic field of the series connection for a given applied voltage V₀.

The transmit antennae are much smaller than the wavelength of electromagnetic radiation at the operating frequency, f. For example, the wavelength at f=1 MHz is c/f where c is the speed of light, or 3×10⁸ m/sec/10⁶/sec=300 meters. Antennas 20 may therefore be designed to project an AC magnetic field through the volume of space where the receivers are expected to be located. An antenna 20 example that fills in the empty space inside a circular coil is a spiral coil. Multiple antennas 20 may be employed to insure good coupling to receivers within a given volume that may be directed in any spatial orientation.

### Transmit/Receive Coil Power Coupling

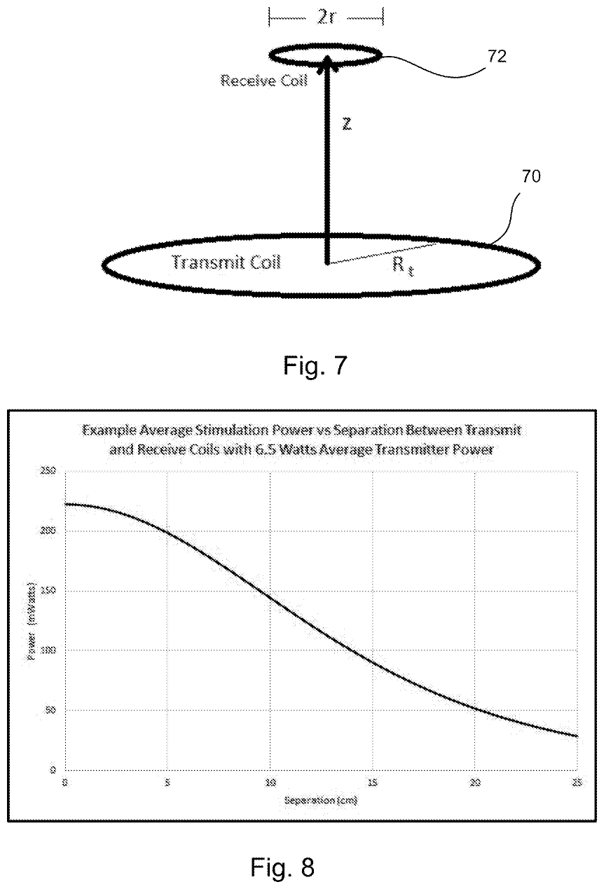

In simple geometries, the power coupled from the transmit coil 70 to the receive coil 72 may be computed exactly. Such calculations provide a proof of concept for the system. A formula for power coupled versus separation of the planes of the two coils defines a practical limit for coil separation. In one example, the transmit coil 70 is circular and lies in one plane, while the receive coil 72 lies on the axis of the transmit coil 70 but is elevated to a plane separated by distance “z” from the transmit coil 70, as shown in FIG. 7. In particular, FIG. 7 provides a simple coil geometry used to compute power coupling efficiency between a transmit coil 70 of radius Rt and a receive coil 72 of radius r, with separation z between transmit 70 and receive coil 72 planes.

An expression for the power coupling efficiency between the transmit coil 70 and receive coil 72 of FIGS. 5 and 7 is given by:

η=κQQ₁u/(1+u)(1+u+κQQ₁) (1)

where η=power coupling efficiency=output power/input power
κ=transformer coupling coefficient
Q=quality factor of the transmit circuit
Q₁=quality factor of the receive circuit

And

u=L₁/(R₁R_{L}C₁)

The quality factors of the transmit and receive circuits are given by:

Q=ωL/R

Q₁=ωL₁/R₁

ω=2πf

And the transformer coupling coefficient is given by:

κ=M/(LL₁)

where M is mutual inductance between the transmit and receive coils. For the coaxial circular coils of FIG. 7 separated by distance z, for which the diameter of the coils is much larger than the diameter of the wire bundle making up the coils, the coupling coefficient is:

κ=(π/8)²(rRₜ/(Rₜ²+z²))³ (2)

The inductances of the transmit and receive planar circular coils are given by:

L=4μ₀RₜN²

L₁=4μ₀rN₁²

Where N and N₁ are the number of turns of wire in the transmit coil 70 and receive coil 72 respectively.

Although the voltage on the storage capacitor 32 in FIG. 5 may be controlled to any lower voltage, an expression for the maximum power and maximum voltage applied to the load is given by:

Pₘₐₓ=V_{L}²/R_{L}=ηV₀²/2R (while stimulation pulse is ON)

V_{L}=V₀SQRT(ηR_{L}/2R) (3)

Which assumes that the voltage V_{L} is applied to the load in alternating polarities to neutralize the stimulation charge. The average power delivered to the load is Pₘₐₓ above times the stimulation duty cycle, which may be 1/10 in an example of 100 pulses delivered per second, each pulse ON for 1 msec.

### Example Calculation

Consider an example in which power is transmitted from a 0.50 m diameter or 0.25 m radius coil wound with 7 turns of #25 gauge Litz wire, having a resistance of 0.1 Ohms per meter, at a frequency of 1 MHz. The total length of wire is 11 meters, and the total resistance is 1.1 Ohms. The transmitter inductance, calculated from the above formula, is 62 μH. At resonance, the capacitance required to tune the transmit circuit to frequency f is:

C=1/(ω²L)

And is equal to C=0.41 nF in this example. The Q of the transmitter is 354.

As an example of a small receiver, consider a 0.025 m diameter circular coil comprising 20 turns of #37 fine wire, having a resistance of 1.6 Ohms per meter. The receive coil resistance is then 2.5 Ohms, and the receive coil inductance is 25 μH. The tuning capacitance is C₁=1.0 nF, and the Q of the receiving coil is Q₁=63.

Consider a typical tissue resistance of R_{L}=1,000 Ohms between stimulating electrodes (not shown) in FIG. 1. The quantity u in Eq (1) is then u=10. The quantity κQQ₁ in the power coupling efficiency in the z=0 plane of the transmitter is then equal to κQQ₁=0.43, and the power coupling efficiency is given by η=0.034, or 3.4%.

The maximum voltage that may be applied to the stimulation electrodes 56a and 56b (FIG. 5) is then given by Eq (3) and is V_{L}=3.9V₀. For example, if V₀=12 volts (24 volt DC power supply), then the maximum stimulation voltage is 47 volts. The input power in this example is 65 Watts when stimulation is ON. The average input power is 6.5 Watts with a 10% duty cycle. The average maximum output power is 0.22 Watts, considered to be at the high end of power needed for stimulation through the skin. This result may be considered a paper proof-of-concept of the stimulation system of FIG. 4.

### Efficiency Versus Separation Between Transmit and Receive Coils

The efficiency in Eq (1) decreases with increasing coil separation according to Eq. (3), which may be re-written as:

F(z)=F(0)/(1+(z/Rₜ)²)³ (4)

And the efficiency as:

η(z)=uF(z)/(1+u)(1+u+F(z)) (5)

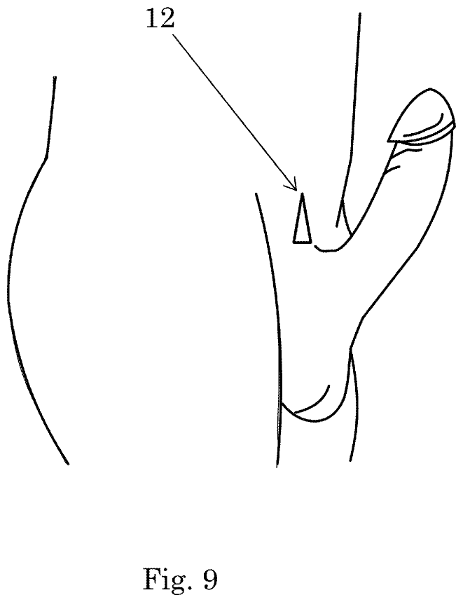

In the above example, F(0)=0.43. FIG. 8 plots the power output of the receive coil in FIG. 7, equal to efficiency in Eq. (5) times power input, as a function of separation between the transmit coil 70 and receive coil 72 for an input power of 6.5 Watts.

It is seen that efficiency is reduced by about a factor of two at a separation equal to one half of the transmitter radius in this example. Increasing the average input power from 6.5 Watts to 13 Watts will result in the same average stimulation power for all receive coil elevations from zero to about 12.5 cm. An input power of 13 Watts spread out over a 50 cm diameter coil will not significantly increase the temperature of the transmitter 70. On the other hand, controlling the receiver 72 voltage will provide uniform stimulation independent of separation. In some embodiments it is desirable to have the stimulation power increase with decreasing separation between transmit coil 70 and receive coil 72, which occurs, for example, at 6.5 Watts input power.

In all applications for which the diameter of the transmit coil 70 is less than 50 cm in the above example, efficiency will be larger than shown in FIG. 8 at small separations of the receive coil 72, but will fall off more quickly with increasing separation. It will be appreciated that more complex windings, for example a spiral winding, a serpentine winding, or multiple windings can produce a more uniform stimulation power across even larger volumes than in FIG. 7.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. Moreover, features or aspects of various example embodiments may be mixed and matched (even if such combination is not explicitly described herein) without departing from the scope of the invention.

What is claimed is:

1. A couple's sexual wellness therapy system configured to be worn by at least two users, the system comprising:
a sexual wellness device configured to be worn by a first of the two users, the sexual wellness device comprising:
at least one action mechanism incorporated therein;
a control garment remote from the sexual wellness device, the control garment configured to be worn by a second of the two users, the control garment being configured to control an action of the action mechanism; and
a system controller in communication with the sexual wellness device and the control garment to control at least one parameter of the sexual wellness system.

2. The couple's sexual wellness therapy system of claim 1, wherein the at least one parameter of the sexual wellness system comprises a proximity parameter.

3. The couple's sexual wellness therapy system of claim 1, wherein the at least one parameter of the sexual wellness system comprises an intensity parameter.

4. The couple's sexual wellness therapy system of claim 1, wherein the at least one parameter of the sexual wellness system comprises a mixed parameter.

5. The couple's sexual wellness therapy system of claim 1, wherein the at least one parameter of the sexual wellness system comprises a treatment parameter.

6. The couple's sexual wellness therapy system of claim 2, wherein the proximity parameter is configured, whereby the control garment activates the action mechanism by proximity to the sexual wellness device.

7. The couple's sexual wellness therapy system of claim 1, further comprising a remote charging system configured to remotely charge at least the sexual wellness device.

8. The couple's sexual wellness therapy system of claim 1, further comprising a remote charging system configured to remotely charge at least the control garment.

9. The couple's sexual wellness therapy system of claim 1, further comprising:
a second sexual wellness device configured to be worn by the second user; and
a second control garment configured to be worn by the first user; wherein the second control garment is configured to control an action mechanism of the second sexual wellness device.

10. A method of treating a couple using a sexual wellness system, the method comprising the steps of:
providing a sexual wellness device configured to be worn by a first user, the sexual wellness device comprising:
at least one action mechanism incorporated therein;
providing a control garment configured to be worn by a second user, the control garment being remote from and configured to control an action of the sexual wellness device; and
providing a system controller in communication with the sexual wellness device and the control garment to control at least one parameter of the sexual wellness system.

11. The method of claim 10, wherein the at least one parameter of the sexual wellness system comprises a proximity parameter, whereby the control garment is configured to control the at least one action mechanism when it is in a proximity thereto.

12. The method of claim 10, wherein the at least one parameter of the sexual wellness system comprises a time parameter, whereby the control garment is able to control the at least one action mechanism for a period of time.

13. The method of claim 10, wherein the system controller is configured to communicate with the control garment, whereby the control garment is configured to control the at least one parameter and the at least one action mechanism.

14. The method of claim 10, wherein the at least one parameter comprises a treatment parameter.

15. The method of claim 14, wherein the treatment parameter comprises the control garment activating the action mechanism of the sexual wellness device to stimulate at least the first user.

16. The method of claim 10, further comprising the step of providing a remote charging system configured to remotely charge the sexual wellness device.

17. The method of claim 10, further comprising the step of providing a remote charging system configured to remotely charge the control garment.

18. The method of claim 10, further comprising:
providing a second at least one sexual wellness device configured to be worn by the second user; and
providing a second control garment configured to be worn by the first user; wherein the second control garment is configured to control an action mechanism of the second at least one sexual wellness device.

19. The method of claim 15, wherein the step of stimulating the first user is to treat a condition of premature ejaculation.

20. The method of claim 15, wherein the step of stimulating the first user is to treat to a condition of low libido.
